(19) United States
(12) Patent Application Publication
(10) Pub. No.: US 2017/0265946 A1
(43) Pub. Date: Sep. 21, 2017

(54) SHAPE SENSED ROBOTIC ULTRASOUND FOR MINIMALLY INVASIVE INTERVENTIONS

(71) Applicant: KONINKLIJKE PHILIPS N.V., Eindhoven (NL)

(72) Inventors: BHARAT RAMACHANDRAN, MORGANVILLE, NJ (US); NERIMAN NICOLETTA KAHYA, EINDHOVEN (NL)

(21) Appl. No.: 15/102,885

(22) PCT Filed: Nov. 27, 2014
(86) PCT No.: PCT/IB2014/066378

§ 371 Date: Jun. 9, 2016

Related U.S. Application Data
(60) Provisional application No. 61/916,821, filed on Dec. 17, 2013.

(51) Int. Cl. A61B 34/20; A61B 1/00; A61B 34/30; A61B 8/00; A61B 8/08

(52) U.S. Cl. CPC A61B 34/20; A61B 8/4254; A61B 8/085; A61B 34/30; A61B 1/00147; A61B 1/00013; A61B 2034/2061; A61B 2034/301; A61B 2090/378

(57) ABSTRACT

A shape sensing system includes a plurality of shape sensing enabled medical devices (118) each having at least one fiber (122). The system is preferably a system for shape sensed robotic ultrasound comprising an endoscope, an ultrasound probe, a medical device and a robot. An optical sensing module (130) is configured to receive optical signals from the at least one optical fiber and interpret the optical signals to provide shape sensing data for each of the plurality of shape sensing enabled medical devices. A registration module (134) is configured to register the plurality of shape sensing enabled medical devices together using the shape sensing data.

### BACKGROUND

Technical Field

This disclosure relates to medical instruments and more particularly to shape sensed ultrasound for minimally invasive interventions.

Description of the Related Art

In certain minimally invasive procedures, such as partial nephrectomy and prostatectomy, ultrasound (US) is used to identify the margin between the healthy and tumorous tissue. The US probe is rather bulky and is typically mounted on a robotic arm to scan the anatomical area ahead in order to discriminate between healthy and tumorous tissue. Afterwards, the probe is moved away from the area of interest. The surgeon will memorize the anatomical location of interest identified through the US probe and will mentally locate the spot in the endoscopic view. This allows the surgeon to navigate with the surgical tools in the endoscopic view and guide the removal of the tumor. However, this mental integration of information requires long training and is prone to errors.

### SUMMARY

In accordance with the present principles, a shape sensing system includes a plurality of shape sensing enabled medical devices each having at least one fiber. An optical sensing module is configured to receive optical signals from the at least one optical fiber and interpret the optical signals to provide shape sensing data for each of the plurality of shape sensing enabled medical devices. A registration module is configured to register the plurality of shape sensing enabled medical devices together using the shape sensing data.

A workstation includes a processor and a memory device coupled to the processor. The memory is configured to store an optical sensing module configured to receive optical signals from at least one optical fiber and interpret the optical signals to provide shape sensing data for each of a plurality of shape sensing enabled medical devices and a registration module configured to register the plurality of shape sensing enabled medical devices together using the shape sensing data.

A method includes providing a plurality of shape sensing enabled medical devices for a subject. Shape sensing data is computed for each of the plurality of shape sensing enabled medical devices. The plurality of shape sensing enabled medical devices is registered together using the shape sensing data.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a shape sensing system, configuration in accordance with one illustrative embodiment;

FIG. 2 shows a display including an endoscopic view and ultrasonic view, in accordance with one illustrative embodiment;

FIG. 3 shows an ultrasound probe fitted with an optical shape sensing sleeve, in accordance with one illustrative embodiment;

FIG. 4 shows an ultrasound probe having at least one fiber secured using shrink tubing, in accordance with one illustrative embodiment;

FIG. 5 shows an ultrasound probe having one or more fibers coupled to the head, in accordance with one illustrative embodiment; and

FIG. 6 is a block/flow diagram showing a method a shape sensed procedure, in accordance with one illustrative embodiment.

### DETAILED DESCRIPTION OF EMBODIMENTS

In accordance with the present principles, systems and methods for shape sensed robotic ultrasound for minimally invasive interventions are provided. One or more medical devices, such as, e.g., an ultrasound probe and endoscope, are integrated with optical shape sensing. Shape sensing may be integrated with the one or more medical devices by securing at least one fiber to the one or more medical devices using, e.g., a sleeve, shrink tubing, a channel within the probe, patch attachment, etc. Based on the shape sensing data, a registration is performed between the one or more medical devices. Registration may be, e.g., landmark-based, fixture-based, image-based, etc. In some embodiments, the one or more medical devices are coupled to one or more moveable features of a configurable device or robot for robotic guidance. The one or more moveable feature may also be integrated with shape sensing such that their relative positions are known.

During a procedure (e.g., partial nephrectomy, prostatectomy, etc.), a shape sensing enabled ultrasound probe and endoscope may be employed. The ultrasound probe may be used to scout ahead to discriminate between healthy and tumorous tissue. Once a tumorous tissue is identified, the endoscope is to be navigated to that location. The registration based on shape sensing of the ultrasound probe and endoscope permits their relative location to be known, providing the surgeon a roadmap to the tumor location. Further, registration based on shape sensing allows for the display of ultrasound images superimposed over or juxtaposed with the endoscopic view, at least in part. This results in accurate targeting of areas of interest, an easy to understand visualization for the operator, and shortened procedure times with potentially improved technical success and clinical outcomes.

It also should be understood that the present invention will be described in terms of medical instruments; however, the teachings of the present invention are much broader and are applicable to any fiber optic instruments. In some embodiments, the present principles are employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk read/write (CD-R/W), Blu-Ray™ and DVD.

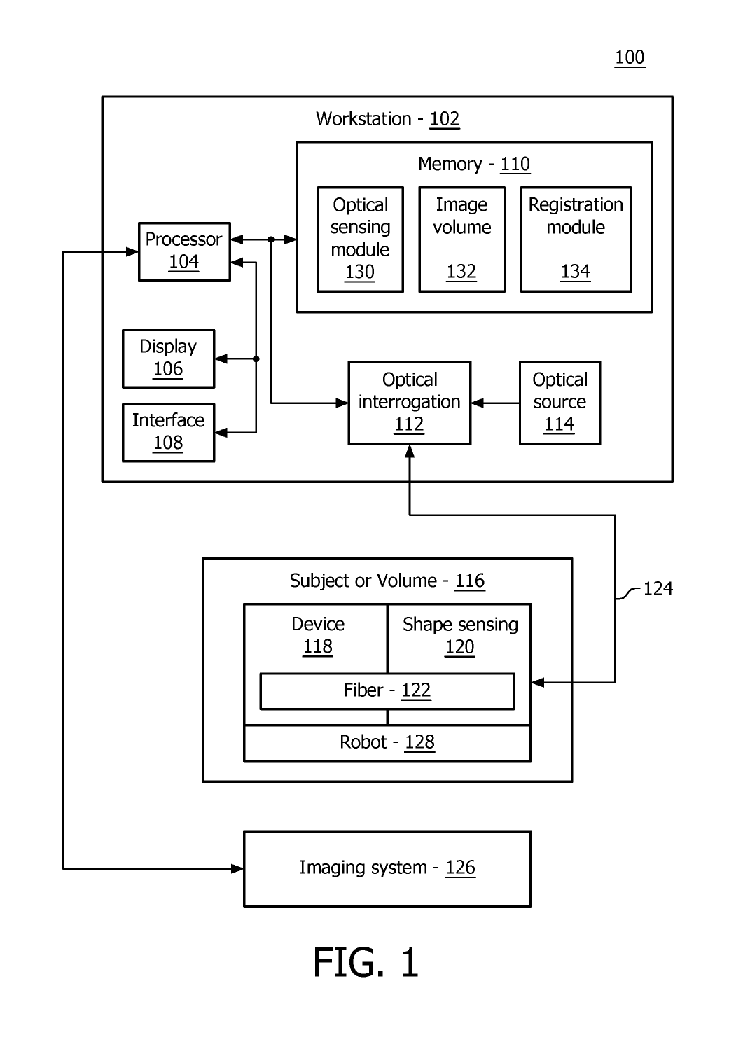

Referring now to the drawings in which like numerals represent the same or similar elements and initially to FIG. 1, a system 100 for shape sensed robotic ultrasound is illustratively shown in accordance with one embodiment. The system 100 may include a workstation or console 102 from which a procedure is supervised and/or managed. Workstation 102 preferably includes one or more processors 104 and memory 110 for storing programs, applications and other data. It should be understood that the function and components of system 100 may be integrated into one or more workstations or systems.

Workstation 102 may include a display 106 for viewing internal images of a subject. Display 106 may also permit a user to interact with the workstation 102 and its components and functions. This is further facilitated by a user interface 108 which may include a keyboard, mouse, a joystick or any other peripheral or control to permit user interaction with the workstation 102.

A shape sensing system includes optical sensing unit/module 130 and a shape sensing device 120 mounted on or integrated into the device 118. The optical sensing module 130 is configured to interpret optical feedback signals from a shape sensing device or system 120 for optical shape sensing (OSS). Optical sensing module 130 is configured to use the optical signal feedback (and any other feedback, e.g., electromagnetic (EM) tracking) to reconstruct deformations, deflections and other changes associated with one or more medical devices or instruments 118 and/or its surrounding region. This permits the determination of strains or other parameters, which will be used to interpret the shape, orientation, etc. of the device 118. The device 118 may include one or more interventional devices, such as a probe, an imaging device, an endoscope, a catheter, a guidewire, a robot, an electrode, a filter device, a balloon device, or other medical devices or components, etc.

The shape sensing system includes an optical interrogator 112 that provides selected signals and receives optical responses. An optical source 114 may be provided as part of the interrogator 112 or as a separate unit for providing light signals to the shape sensing device 120. Shape sensing device 120 includes one or more optical fibers 122 which are coupled to the device 118 in a set pattern or patterns. The optical fibers 122 are configured to exploit their geometry for detection and correction/calibration of a shape of the device 118. Optical sensing module 130 works with optical sensing module 115 (e.g., shape determination program) to permit tracking of instrument or device 118. The optical fibers 122 connect to the workstation 102 through cabling 124. The cabling 124 may include fiber optics, electrical connections, other instrumentation, etc., as needed.

Shape sensing system 120 with on fiber optics may be based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric mirror. A fiber Bragg grating can therefore be used as an inline optical filter to block certain wavelengths, or as a wavelength-specific reflector.

A fundamental principle behind the operation of a fiber Bragg grating is Fresnel reflection at each of the interfaces where the refractive index is changing. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optical sensors. In an FBG sensor, the object being measured (e.g., strain) causes a shift in the Bragg wavelength.

One advantage of this technique is that various sensor elements can be distributed over the length of a fiber. Incorporating three or more cores with various sensors (gauges) along the length of a fiber that is embedded in a structure permits a three dimensional form of such a structure to be precisely determined, typically with better than 1 mm accuracy. Along the length of the fiber, at various positions, a multitude of FBG sensors can be located (e.g., 3 or more fiber sensing cores). From the strain measurement of each FBG the curvature of the structure can be inferred at that position. From the multitude of measured positions, the total three-dimensional form is determined.

As an alternative to fiber-optic Bragg gratings, the inherent backscatter in conventional optical fiber can be exploited. One such approach is to use Rayleigh scatter in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multi-core fiber, the 3D shape and dynamics of the surface of interest can be followed. Enhanced Rayleigh scatter can also be employed. Enhanced Rayleigh scatter is similar to Rayleigh scatter but instead of the inherent backscatter, the level of impurity in the fiber is increased, resulting in a higher signal.

The one or more devices 118 preferably include a plurality of devices 118 including imaging devices and surgical devices. In a preferred embodiment, the imaging devices include an ultrasound probe and an endoscope, which may be part of one or more imaging systems 126. Other devices 118 or imaging devices may also be employed in various combinations, such as, e.g., two endoscopes, two ultrasound probes, a shape (volume coated with shape at an instant or over time) with an ultrasound probe or video image, etc. The devices 118 may be employed to discover or observe a target in the subject 116 by collecting imaging data during a procedure to create an imaging volume 132. The target may include any area of interest, such as a lesion, an injury site, a functioning organ, etc. on or in the subject 116. The images 132 from each imaging device may be taken at a same time or at different times. In one example, the ultrasound probe may be a two-dimensional probe, three-dimensional probe (e.g., the Philips™ S8-3t microTEE probe), or four-dimensional probe (i.e., three-dimensional plus time). The choice of the probe may be based upon the clinical application.

Preferably, each of the plurality of devices 118 is integrated with shape sensing 120 such that the plurality of devices 118 is OSS-enabled. Shape sensing 120 may be integrated into devices 118 by: (1) fitting an OSS sleeve over the body of the device 118; (2) placing OSS fiber 122 within a channel inside the device 118; (3) coupling OSS fiber 122 at the head of the device 118 using, e.g., tape/patch attachment, etc.; and (4) OSS fiber 122 within shrink tubing over the length of the device 118, in part or in full. Other means of integrating shape sensing system 120 with the devices 118 may also be employed within the context of the present invention to provide an OSS-enabled device.

A registration module 134 may be employed to register the plurality of devices 118 with each other using shape sensing data. In a preferred embodiment, the plurality of devices 118 includes an OSS-enabled ultrasound probe, an OSS-enabled endoscope, and an OSS-enabled surgical device and the registration module 134 may be configured to register the ultrasound, endoscope and surgical information together. This creates a roadmap for the user (e.g., surgeon) allowing for an improved workflow. Registration may be landmark-based, fixture-based, and image-based. Other methods of registration may also be employed within the context of the present principles. In a particularly useful embodiment, registration of an OSS-enabled imaging device to an OSS-enabled medical device is continuously updated (e.g., in real-time, at set intervals, etc.) to thereby provide a dynamically updated roadmap to the surgeon as the procedure is being performed.

In one embodiment, registration module 134 performs landmark-based registration. Known positions of landmarks (e.g., fiducial markers, anatomical reference points in the subject 116, etc.) are employed as reference positions. A first OSS-enabled device 118 is moved to 3 or more reference positions in the field of view of the other OSS-enabled devices for three-dimensional imaging (2 or more reference positions may be possible for two dimensions). For example, an OSS-enabled ultrasound probe may be moved to 3 reference positions in the endoscope field of view, or the OSS-enabled endoscope may be moved to 3 reference positions in the ultrasound field of view. In a particularly useful embodiment, each of the OSS-enabled devices 118 are moved to 3 or more reference positions in the field of view of the other OSS-enabled devices, which provides for built-in redundancy for optimization.

In another embodiment, registration module 134 performs fixture-based registration. Each OSS-enabled device 118 is placed within a fixture. The fixture is then moved in a known manner. In one embodiment, the devices 118 are placed in a same fixture at different times (e.g., one after another) for each device 118. In another embodiment, the devices 118 are placed in different fixtures sa e time or at different times). The movement of each fixture is known by, e.g., having a known path or having either a known velocity or acceleration. Based on the relationship between the paths, the location of the devices 118 is known with respect to each other.

In still another embodiment, registration module 134 performs image-based registration. An imaging device (e.g., X-ray) may capture the OSS-enabled device 118 and the OSS may be matched to the position of the device 118 in the X-ray. Similarly, an ultrasound probe may be matched to the X-ray and the endoscope may be matched to the X-ray to determine the relative pose and orientation of devices for image-based registration. This imaging information may be employed to correct for the perceived position and orientation of the devices 118.

In one particularly useful embodiment, the workstation 102 may optionally include a robot 128. The robot 12.8 may include a configurable device or robot having movable feature(s). The moveable feature(s) may include arms including linkages, appendages, joints, etc. Arms of the robot 128 may be coupled with one or more devices 118, which allows for the robot 128 to actuation the devices 118 in a controlled fashion. In theory, the relative pose and orientation of the robot 128 should be decipherable from the kinematic movement of the moveable feature(s). However, this is very difficult due to mechanical tolerances and control at the tip (e.g., a 2 mm translation at the proximal region needn't manifest itself in exactly the same at the distal potion). It is sometimes not possible to know exactly where the distal tip of the robotic device is based on the voltage applied or the proximal force control.

Preferably, the devices 118 and/or arms of the robot 128 are integrated with shape sensing 120 such that the relative position of each arm is known based on both the position and the movement of the robot. Employing OSS will allow the motion of all devices to be recorded in a single coordinate system, that of the OSS. As a result, dynamic motion of each of the plurality of devices 118 (e.g., ultrasound probe, endoscope, surgical device, etc.) can be recorded. The robot 128 may be an open-loop robot and a closed-loop robot using feedback from the OSS.

During a procedure (manual or robotic), shape sensing data from shape sensing device 120 is collected for the OSS-enabled device 118 (e.g., ultrasound probe and endoscope) for registration. Since the surgeon tracks the motion of the OSS-enabled device 118, the exact location of the tumor is known for removal. A display 106 and/or user interface 108 may be employed to display ultrasound images of locations of interest from the endoscopic view. This may include overlaying at least portion of the ultrasound images over the endoscopic view at, e.g., landmarks, regions of interest, etc. Intra-operative correction and motion compensation (e.g., from a heartbeat, breathing, etc.) can be performed to account for the same in the images (e.g., deformations due to breathing can be measured using shape sensing).

In one embodiment, an OSS-enabled imaging device may be moved around in the subject 116 and, by tracking its position with OSS, a larger field of view can be stitched together, allowing for a better visualization of the target area. In another embodiment, an operator may drop landmarks or other points of interests or useful pieces of information identified in a first imaging device (e.g., ultrasound imaging) into a second imaging device (e.g., endoscopic view) for visualization in real-time as the operator proceeds. For example, in ultrasound, an operator may observe a boundary between benign and malignant tissue. A few landmarks or line may be selected and these points/lines may be shown in the endoscopic view (e.g., overlaid or side-by-side). In another example, a robot 128 may be employed to perform a procedure (e.g., scissor or canter) based on the selected line. Immediately after the procedure, an OSS-enabled ultrasound probe 118 can be used to confirm that the procedure was successful (e.g., the target tumor has been removed). By employing the shape sensing system 120, the surgeon can quickly and easily navigate to the target location and, if needed, repeat the procedure.

In some embodiments, pre-operative information can be registered with the visualization of the imaging device 118 (e.g., endoscopic visualization). The pre-operative imaging may be performed at another facility, location, etc. in advance of any procedure. OSS may be employed to create virtual endoscopic view, thus allowing the surgeon to perform the procedure safer and faster. The virtual image may be a rendering of what the :15 real image (e.g., from an endoscope) may look like based on previously acquired data, such as, e.g., computerized tomography (CT scan), cone-beam CT, magnetic resonance imaging (MRI), ultrasound, etc.

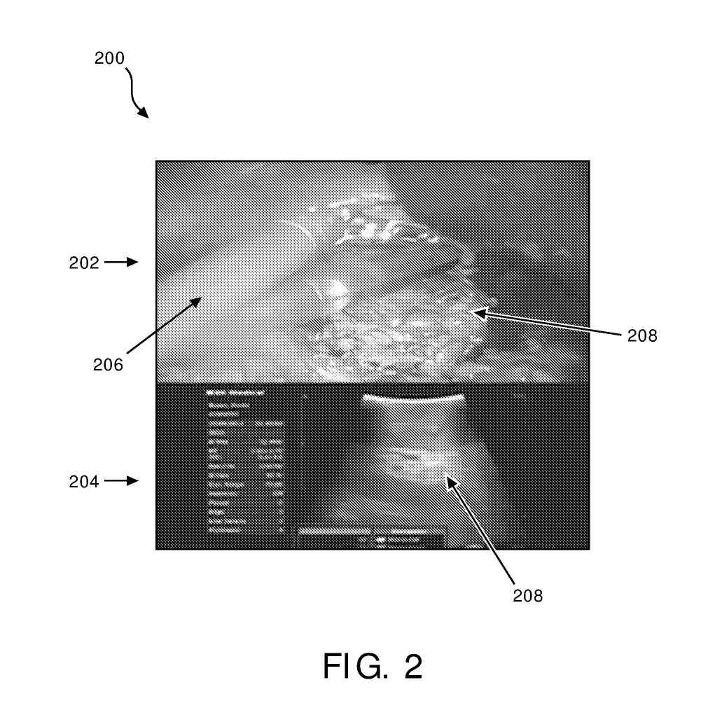

Referring now to FIG. 2, a display 200 shows an endoscopic view 202 and ultrasonic view 204 during a procedure (e.g., partial nephrectomy), in accordance with one illustrative embodiment. Ultrasonic view 204 scans the anatomical area ahead to discriminate between healthy and tumorous tissue. A tumor 208 is identified in the ultrasonic view 204. The endoscopic device and ultrasonic device are OSS-enabled to permit registration between the devices. This allows a surgical device 206 to be manually or robotically guided to the location of the tumor 208 in the endoscopic view 202. By registering the OSS-enabled devices, a roadmap for the surgeon to the target area may be created to improve workflow. In some embodiments, the endoscopic view 202 may include an overlay of the ultrasonic view 204, at least in part (e.g., tumor 208).

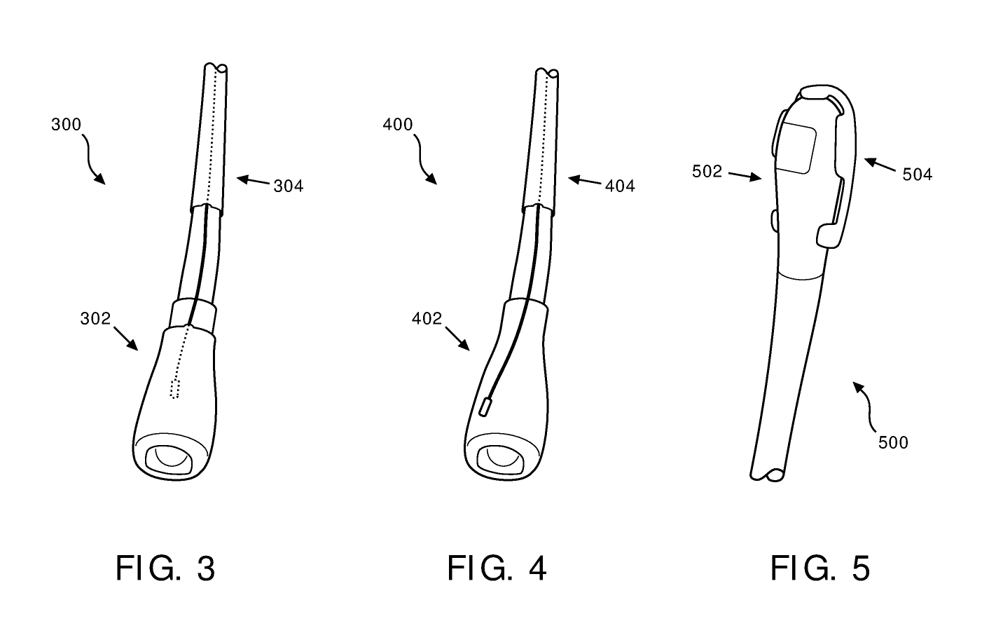

Referring now to FIG. 3, an OSS-enabled ultrasound probe 300 is shown in accordance with one illustrative embodiment. The ultrasound probe 302 is integrated with optical shape sensing by fitting an OSS sleeve 304 over a length of the probe 302. The sleeve 304 secures fibers along the probe 302 for shape sensing. It should be understood that the sleeve 304 may include any structure configured to fit around the fibers and the length of the probe 302 such that the fibers are secured to probe 302.

Referring now to FIG. 4, an OSS-enabled ultrasound probe 400 is shown in accordance with one illustrative embodiment. The ultrasound probe 402 is integrated with optical shape sensing using shrink tubing 404. The fiber may be placed in a small tube along at least a portion of the length of the probe 402. Once positioned in the tube, shrink tubing 404 is applied to secure the tube to the probe 402 for shape sensing. Heat may be applied to the shrink tubing 404 such that it fits securely around the fibers and probe 402,

Referring now to FIG. 5, an OSS-enabled ultrasound probe 500 is shown in accordance with one illustrative embodiment. The ultrasound probe 502 is integrated with optical shape sensing by coupling fibers to a head of the probe 502 using a tape/patch attachment 504. In one embodiment, the tape/patch attachment 504 is employed to secure the fiber to the head of the probe 502 (which could be a point or a few millimeters). The remaining portions of the fiber remains unsecured to the probe 502, which allows the fiber to account for path length change. In another embodiment, the tape/patch attachment 504 is secured to the head of the probe 502 as well as a proximal section of the length of the probe 502. In this embodiment, a buffer loop may be provided to compensate for path length change. Other approaches to coupling fiber to the head of the probe 502 may also be employed, such as, e.g., tape, adhesive, etc.

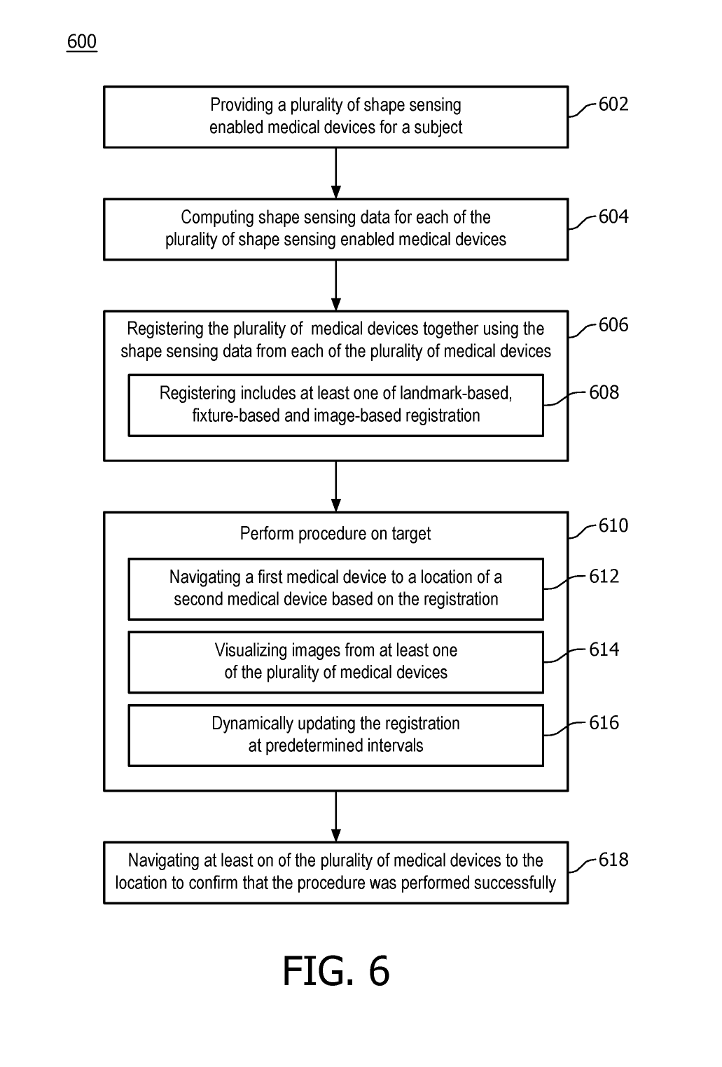

Referring now to FIG. 6, a block/flow diagram showing a method for shape sensed robotic ultrasound is depicted in accordance with one illustrative embodiment. In block 602, a plurality of shape sensing enabled medical devices is provided for around a subject. Preferably, the plurality of medical devices includes a shape sensing enabled ultrasound probe, an endoscope and interventional medical device. The shape sensing may be integrated to the medical devices by securing one or more fibers into the plurality of medical devices by using, e.g., an OSS sleeve, shrink tube, etc., placing the one or more fibers in a channel of a medical device, coupling (tape or patch attachment) the one or more fibers to a head of a medical device, etc. Other methods of integrating shape sensing may also be employed. In one embodiment, the plurality of medical devices may be coupled to a configurable device, such as a robot, having one or more movable features (e.g., linkages, appendages, joints). The one or more movable features may be integrated with shape sensing.

In block 604, shape sensing data from each of the plurality of shape sensing enabled medical devices are computed. In block 606, the plurality of medical devices are registered together based on the shape sensing data from each of the plurality of medical devices such that a relative position of each of the plurality of medical devices is known. In block 608, registering may include at least one of landmark-based, fixture-based and image-based registration. Landmark-based registration includes positioning a medical device to 3 or more known positions within a field of view with the other medical devices. Fixture-based registration includes placing each of the plurality of medical devices in a fixture. The same fixture may be employed at different times or different fixtures may be employed. The fixtures are moved in a known manner, i.e., in a known path or with either known velocities or accelerations. The relative location of the medical devices is known based on the relationship between the paths. Image-based registration includes comparing imaging data from the plurality of medical devices to determine a relative position and orientation of the medical devices.

In block 610, a procedure is performed on a target. In block 612, performing the procedure includes navigating a first medical device to a location of a second medical device based on the registration. The location may be the location of the target. In block 614, images of the plurality of medical devices may be visualized based upon the known relative locations of the plurality of medical devices. Visualizing may include overlaying or juxtaposing images from a first medical device, at least in part, onto images of a second medical device. Visualizing may also include stitching together multiple fields of view of a medical device to provide a larger field of view. Visualizing may further include compensating motion from the subject (e.g., due to breathing) in the visualization. In block 616, the registration may be dynamically updated during the procedure. In block 618, after the procedure is complete, a medical device may be navigated to the location to confirm that the procedure was successfully performed.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for shape sensed robotic ultrasound for minimally invasive interventions (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims,

1. A shape sensing system, comprising:
a plurality of shape sensing enabled medical devices each having at least one fiber;
a robot having one or more shape sensing enabled movable features that are coupled to the plurality of shape sensing enabled medical devices and is configured to actuate the plurality of shape sensing enabled medical devices;
an optical sensing module configured to receive optical signals from the at least one optical fiber and the shape sensing enabled movable features of the robot and interpret the optical signals to provide shape sensing data for each of the plurality of shape sensing enabled medical devices and the robot; and
a registration module configured to register the plurality of shape sensing enabled medical devices and the robot together using the shape sensing data.

2. The system as recited in claim 1, wherein the registration module is configured to register the plurality of shape sensing enabled medical devices together by positioning at least one of the plurality of shape sensing enabled medical devices to a known position within a field of view of remaining plurality of shape sensing enabled medical devices.

3. The system as recited in claim 1, wherein the registration module is configured to register the plurality of shape sensing enabled medical devices together by placing each of the plurality of shape sensing enabled medical devices in a fixture and moving the fixture in a known manner.

4. The system as recited in claim 1, wherein the registration module is configured to register the plurality of shape sensing enabled medical devices together by comparing images from each of the plurality of shape sensing enabled medical devices.

5. The system as recited in claim 1, wherein the plurality of shape sensing enabled medical devices include at least one fiber secured to a medical device by at least one of: a shape sensing sleeve including the at least one fiber fit around the medical device, the at least one fiber placed within a channel in the medical device, the at least one fiber coupled to a head of the medical device, and the at least one fiber secured to the medical device by shrink tubing.

6. (canceled)

7. The system as recited in claim 1, wherein the robot comprises a closed-loop robot using the shape sensing data as feedback.

8. (canceled)

9. The system as recited in claim 1, wherein the registration module is configured to update registration at predefined intervals.

10. The system as recited in claim 1, wherein the plurality of shape sensing enabled medical devices include an endoscope, an ultrasound probe and a medical device and further wherein a robot is configured to navigate the medical device to a location of an imaging view of the endoscope based upon registered input from the ultrasound probe to perform a procedure.

11. A workstation, comprising:
a processor;
a memory device coupled to the processor and configured to store:
an optical sensing module configured to receive optical signals from at least one optical fiber and interpret the optical signals to provide shape sensing data for each of a plurality of shape sensing enabled medical devices; and a robot having shape sensing enabled movable features that are coupled to the plurality of shape sensing enabled medical devices: and
a registration module configured to register the plurality of shape sensing enabled medical devices and the robot together using the shape sensing data.

12. The workstation as recited in claim 11, wherein the registration module is configured to register the plurality of shape sensing enabled medical devices together when at least one of the plurality of shape sensing enabled medical devices to a known position within a field of view of remaining plurality of shape sensing enabled medical devices.

13. The workstation as recited in claim 11, wherein the registration module is configured to register the plurality of shape sensing enabled medical devices together when each of the plurality of shape sensing enabled medical devices in a fixture and moving the fixture in a known manner.

14. The workstation as recited in claim 11, wherein the registration module is configured to register the plurality of shape sensing enabled medical devices together by comparing images from each of the plurality of shape sensing enabled medical devices.

15. The workstation as recited in claim 11, wherein the workstation further comprises a plurality of shape sensing enabled medical devices said plurality of shape sensing enabled medical devices including at least one fiber secured to a medical device by at least one of: a shape sensing sleeve including the at least one fiber fit around the medical device, the at least one fiber placed within a channel in the medical device, the at least one fiber coupled to a head of the medical device, and the at least one fiber secured to the medical device by shrink tubing.

16. A method, comprising:
providing a plurality of shape sensing enabled medical devices for a subject;
providing a robot having one or more shape sensing enabled movable features coupled to the plurality of shape sensing enabled medical devices;
computing shape sensing data for each of the plurality of shape sensing enabled medical devices and the robot; and
registering the plurality of shape sensing enabled medical devices and the robot together using the shape sensing data.

17. The method as recited in claim 16, wherein registering the medical devices includes positioning at least one of the plurality of shape sensing enabled medical devices to a known position within a field of view of remaining plurality of shape sensing enabled medical devices.

18. (canceled)

19. (canceled)

20. (canceled)

21. (canceled)

22. (canceled)

23. (canceled)

24. (canceled)

25. (canceled)
